(19) United States
(12) Patent Application Publication
(10) Pub. No.: US 2015/0283252 A1
(43) Pub. Date: Oct. 8, 2015

(54) STABLE PHARMACEUTICAL COMPOSITION OF PEGINTERFERON ALPHA-2B

(71) Applicant: LUPIN LIMITED, Mumbai (IN)

(72) Inventors: Paresh Natwarlal Vadgama, Pune (IN); Anjali Deepak Apte-Deshpande, Pune (IN); Rustom Sorab Mody, Pune (IN)

(21) Appl. No.: 14/438,394

(22) PCT Filed: Oct. 25, 2013
(86) PCT No.: PCT/IB2013/059657

§ 371 Date: Apr. 24, 2015

(30) Foreign Application Priority Data
Oct. 26, 2012 (IN) 1234/KOL/2012

(51) Int. Cl. A61K 47/40; A61K 47/48; A61K 47/02; A61K 47/32; A61K 47/26; A61K 47/22; A61K 47/18; A61K 38/21; A61K 47/12

(52) U.S. Cl. CPC A61K 47/40; A61K 38/212; A61K 47/48215; A61K 47/02; A61K 47/12; A61K 47/26; A61K 47/22; A61K 47/183; A61K 47/32

(57) ABSTRACT

The present invention relates to the stable pharmaceutical compositions comprising PEG-interferon alpha-2b. More particularly, it relates to the stable pharmaceutical compositions comprising PEG-interferon alpha-2b and cryoprotectant selected from the group consisting of 2-Hydroxy propyl beta-cyclodextrin, sucralose, or polyvinylpyrrolidone 4000. It also relates to the methods of manufacturing the composition, method of administration and kits containing the same.

### FIELD OF INVENTION

The invention provides stable pharmaceutical compositions comprising of PEG-interferon alpha-2b. The invention also provides methods of manufacturing the composition, method of administration and kits containing the same.

### BACKGROUND OF INVENTION

Interferons are cytokines secreted by all eukaryotic cells in response to the infection by pathogens like bacteria, viruses or parasites. Hence, these proteins have therapeutic potential for variety of infections mainly viral infections and proliferative disorders like cancers (Pfeffer et al. 1998 Cancer Research 58, 2489-2499).

Human interferons are classified into 3 types based on their cellular origin and antigenicity: alpha-interferon (leukocyte), beta-interferon (fibroblasts) and gamma-interferon (Bcells) (U.S. Pat. No. 7,632,491). Recombinant alpha interferons were first approved over two decades ago by US FDA for the treatment of hairy cell leukemia. Since then different types of recombinant interferons are commercially available for the treatment of many diseases like chronic hepatitis C, malignant melanoma, non-Hodgkin's lymphoma etc. (Pfeffer et al. 1998 Cancer Research 58, 2489-2499 and Wang et al. 2002 Advanced Drug Delivery Reviews 54, 547-570).

However, like many other parenterally administered proteins, they have some limitations in their use due to antigenicity which lead to the formation of neutralizing antibody and short pharmacological half-life which consequently leads to administering repeated dosage to achieve desired blood levels (U.S. Pat. No. 6,180,096). This problem can be overcome by conjugating these proteins to polymers like polyethylene glycol (PEG). PEG is a non-immunogenic and non-toxic polymer. Additionally, it is soluble in water and several organic solvents. When a protein is chemically conjugated to PEG moiety the water solubility of the protein increases (US 700314). Pegylation can improve the pharmacokinetic properties of the molecule, give thermal and physical stability, protect against enzymatic degradation, and increase in-vivo circulating half-life due to decreased clearance from the body. However, the selection of right size of PEG molecule, protein-PEG ratio and pegylation process parameters are crucial for pegylation process and getting biologically active protein molecule (Bailon et al, 1998 Pharm. Sci. Technology Today Vol. 1, No. 8, 352-356).

The stability of such conjugates can be achieved by right composition which can maintain the conjugated protein in stable form and removal of water from composition by techniques like Freeze drying/lyophilization (US20100074865).

U.S. Pat. No. 5,730,969 discloses a protein composition comprising an effective stabilizing amount of cyclodextrin.

U.S. Pat. No. 7,846,427, U.S. Pat. No. 6,180,096, U.S. Pat. No. 6,250,469, U.S. Pat. No. 5,766,582 and U.S. Pat. No. 7,632,491 disclose pharmaceutical compositions comprising PEG-interferon.

PCT applications WO 2010064258, WO 200135987, WO2008062481 and WO2004096263 disclose interferon composition.

The present invention is related to a stable composition comprising Pegylated interferon alpha-2b conjugate.

### SUMMARY OF THE INVENTION

In an embodiment, the invention is related to stable pharmaceutical composition comprising a biologically active PEG-interferon alpha-2b (PEG-IFN α-2b) and a cryoprotectant selected from the group consisting of 2-Hydroxy propyl beta-cyclodextrin (HPBCD), sucralose, and polyvinylpyrrolidone 4000 (PVP 4000).

In another embodiment, the invention is related to a stable pharmaceutical composition comprising PEG-IFN α-2b, cryoprotectant selected from the group consisting of HPBCD, sucralose and PVP 4000, and buffer.

In yet another embodiment, the invention is related to a stable pharmaceutical composition having a pH in the range of 4.0 to 8.0 which comprises PEG-IFN α-2b, cryoprotectant selected from the group consisting of HPBCD, sucralose and PVP 4000, and buffer selected from the group comprising of sodium or potassium phosphate, citrate, L-Histidine and L-Arginine hydrochloride and combinations thereof.

In yet another embodiment, the invention is related to the stable pharmaceutical composition further comprising one or more surfactants.

In yet another embodiment, the invention is related to the stable pharmaceutical composition further optionally comprising one or more tonicity agents to maintain the tonicity of the pharmaceutical composition.

In yet another embodiment, the invention is related to a process of preparation of the stable pharmaceutical composition of the present invention.

In yet another embodiment, the invention is related to the method of treating a disease in human using the stable pharmaceutical composition of the present invention. The disease may be hepatitis C, hepatitis B or melanoma with microscopic or gross nodal involvement within 84 days of definitive surgical resection including complete lymphadenectomy.

The details of one or more embodiments of the invention set forth in the below are illustrative in nature only and not intended to limit to the scope of the invention. Other features, objects and advantages of the inventions will be apparent from the description and claims.

### DETAIL DESCRIPTION OF INVENTION

The invention provides a stable pharmaceutical composition comprising PEG-IFN α-2b and cryoprotectant selected from the group consisting of HPBCD, sucralose, and PVP 4000. More particularly the stable pharmaceutical composition is sterile and ready for parenteral administration.

The present pharmaceutical composition comprises a purified PEG-IFN α-2b and cryoprotectant selected from the group consisting of HPBCD, sucralose and PVP 4000, buffer, surfactant, tonicity modifier and other excipients in suitable combination thereof.

The stable pharmaceutical composition of the present invention is packaged in a vial, prefilled syringe or cartridge. The preferred packaging is vial.

In an embodiment of the invention, PEG₁₂₀₀₀ interferon alpha-2b is used which is obtained from recombinant DNA technology using E. coli cells. The concentration of the PEG-IFN α-2b is from 0.03 mg/ml to 2 mg/ml.

In another embodiment of the invention, PEG-IFN α-2b composition comprises a cryoprotectant selected from the group consisting of HPBCD, sucralose, and PVP 4000. The concentration of the cryoprotectant varies from about 10-250 mg/ml.

In yet another embodiment of the invention, the buffer is selected from a group of phosphate-citrate buffer, phosphate buffer, citrate buffer, histidine acetate, histidine-histidine hydrochloride, L-Histidine, L-Argenine hydrochloride, bicarbonate buffer, succinate buffer, citrate buffer, TRIS buffer, either alone or in combination. The preferred buffers of the invention are phosphate buffer, citrate buffer, phosphate-citrate buffer, L-Histidine or L-Ariginine hydrochloride. The concentration of the buffer in the solution is 5 mM to 100 mM with individual buffer component has molar concentration range between 1-100 mM.

In yet another embodiment of the invention, the buffer system of the present invention maintains the pH of the composition in the range of 4.0 to 8.0. The preferred pH is 6.4 to 7.2.

In yet another embodiment of the invention, the surfactant is selected from the group comprising of polysorbate-based non-ionic surfactants, dodecyl sulfate (SDS), Lecithin either alone or in combination. Further, the polysorbate is selected from polysorbate 20 or polysorbate 80. The preferred surfactant is polysorbate 80. The concentration of the surfactant varies from about 0.01-1.0 mg/ml.

In yet another embodiment of the invention, the stabilized lyophilized composition optionally comprises of a parenterally acceptable tonicity agent. The tonicity agent is selected from a group of salts, for example sodium chloride, potassium chloride, calcium chloride and the saccharides, like for example mannitol, sucrose, glucose and their likes and/or amino acids, for example arginine, cysteine, histidine and the like. The preferred tonicity agent is sodium chloride and mannitol. The more preferred tonicity agent is sodium chloride. The preferred range of the sodium chloride varies from 0-9 mg/ml.

The invention may further comprise other pharmaceutically stable excipients such as preservatives, anti-chelating agents. The excipient may be selected from the group comprising of saccharides selected from the group comprising of mannitol, galactose and maltose; EDTA, urea, phenol, m-cresol, p-cresol, o-cresol.

In an embodiment the invention is a stable lyophilised pharmaceutical composition reconstituted in reconstituting agents. The preferred reconstituting agent is sterile water for injection or sterile saline solution. The stable lyophilised pharmaceutical composition of the present invention is packaged in a vial, prefilled syringe or cartridge. The preferred packaging is vial.

The stable pharmaceutical composition is lyophilized and can be stored for a long period of time at 2-8° C. and for 6 months at 25° C.

The pharmaceutical composition of the present invention is stable at 5° C., 25° C. and 40° C., preferably 5° C. and has a long shelf life for more than 6 months.

In another embodiment of the invention, the composition provided in this invention is a stable Pegylated interferon solution comprising cryoprotectant selected from the group consisting HPBCD, sucralose, and PVP 4000, a phosphate-citrate buffer, polysorbate 80 as surfactant and optionally NaCl as a tonicity agent with a long shelf life.

In another embodiment the composition is a powder, an aqueous composition, or a reconstituted liquid composition.

### EXPERIMENTAL SECTION

The examples which follow are illustrative of the invention and are not intended to be limiting.

recombinant human IFN α-2b was obtained from E. coli cells by rDNA technology, purified using one or more chromatographic steps such as Hydrophobic interaction chromatography or ion exchange chromatography, filtered, and pegylated to obtain PEG-IFN α-2b.

### General Process for Preparing Stable Pharmaceutical Composition of PEG-IFN α-2b

Formulation process for Peg-IFN drug substance comprised of 3 steps viz. preparation of formulated bulk, filling in vials and lyophilization. Formulated bulk was prepared by diluting the drug substance with the formulation buffer to achieve the desired concentration of formulated bulk. The formulation buffer was prepared by adding required quantity of Disodium phosphate dihydrate and Citric acid to WFI followed by mixing. To this solution, required quantities of cryoprotectant and other excipients were added in a stepwise manner and the desired volume was adjusted with WFI after adjustment of pH. The formulation buffer was then aseptically filtered using 0.22μ sterilizing grade PES filter. As per the batch calculation, the required quantity of Peg-IFN (in same composition) was aseptically diluted with the filtered formulation buffer to achieve the desired concentration of Peg-IFN composition.

### Example 1

Formulation process for Peg-IFN drug substance comprised of 3 steps viz. preparation of formulated bulk, filling in vials and lyophilization. Formulated bulk was prepared by diluting the drug substance with the formulation buffer to achieve the desired concentration of formulated bulk. The formulation buffer was prepared by adding required quantity (as mentioned in table 1) of Disodium phosphate dihydrate and Citric acid to WFI followed by mixing. To this solution, required quantities of cryoprotectant HPBCD, Sodium Chloride and polysorbate 80 were added in a stepwise manner and the desired volume was adjusted with WFI after adjustment of pH. The formulation buffer was then aseptically filtered using 0.22μ sterilizing grade PES filter. As per the batch calculation, the required quantity of Peg-IFN (in same formulation) was aseptically diluted with the filtered formulation buffer to achieve the desired concentration of Peg-IFN composition. The formulated bulk was filtered through 0.22μ sterilizing grade PES filter and was aseptically dispensed into the vials. The ranges of the excipients along with the PEG-IFN α-2b is provided in table 1.

TABLE 1
Quantities of respective excipients along with of PEG-IFN α-2b

| Ingredients | Quantity | |
|---|---|---|
| PEG-IFN α-2b | 0.03-2 | mg/mL |
| Buffer | 5-100 | mM |
| Cryoprotectant | 10-250 | mg/mL |
| Tonicity Agent | 0-9 | mg/mL |
| Surfactant | 0.01-1 | mg/mL |

### Example 2

The process for preparing PEG-IFN α-2b composition is as described in Example 1, wherein the cryoprotectant used is HPBCD. The quantities of the excipients along with the PEG-IFN α-2b is provided in table 2.

TABLE 2
Unit formula for the composition of PEG-IFN α-2b in the stability studies

| | | | Molar Conc. |
|---|---|---|---|
| Ingredients | Qty/vial | Conc. | (mM) |
| PEG-IFN α-2b | 0.12 mg | 0.16 mg/mL | 0.005 |
| Sodium Phosphate Dibasic | 2.15 mg | 2.90 mg/mL | 16.46 |
| dihydrate | | | |
| Citric Acid anhydrous | 0.37 mg | 0.50 mg/mL | 2.58 |
| HPBCD | 59.20 mg | 80.00 mg/mL | 57.20 |
| NaCl | 4.44 mg | 6.00 mg/mL | 102.67 |
| Polysorbate 80 | 0.07 mg | 0.10 mg/mL | 0.076 |

### Example 3

The process for preparing PEG-IFN α-2b composition is the same as described in Example 1, wherein the cryoprotectant used is sucralose. The quantities of the excipients along with the PEG-IFN α-2b is provided in table 3.

TABLE 3
Unit formula for the composition of PEG-IFN α-2b in the stability studies

| | | | Molar Conc. |
|---|---|---|---|
| Ingredients | Qty./vial | Conc. | (mM) |
| PEG-IFN α-2b | 0.12 mg | 0.16 mg/mL | 0.005 |
| Sodium Phosphate Dibasic | 2.15 mg | 2.93 mg/mL | 16.46 |
| dihydrate | | | |
| Citric Acid anhydrous | 0.37 mg | 0.34 mg/mL | 2.58 |
| Sucralose | 59.20 mg | 80.00 mg/mL | 201.2 |
| Polysorbate 80 | 0.074 mg | 0.10 mg/mL | 0.0763 |

### Example 4

The process for preparing PEG-IFN α-2b composition is the same as described in Example 1, wherein the cryoprotectant used is sucralose and buffer used is L-Histidine and L-Arginine. The quantities of the excipients along with the PEG-IFN α-2b is provided in table 4.

TABLE 4
Unit formula for the composition of PEG-IFN α-2b in the stability studies

| | | | Molar Conc. |
|---|---|---|---|
| Ingredients | Qty./vial | Conc. | (mM) |
| PEG-IFN α-2b | 0.12 mg | 0.16 mg/mL | 0.005 |
| L-Histidine | 0.85 mg | 1.15 mg/mL | 7.43 |
| L-Arginine hydrochloride | 10.54 mg | 14.24 mg/mL | 67.58 |
| Sucralose | 59.20 mg | 80.00 mg/mL | 201.2 |
| Polysorbate 80 | 0.074 mg | 0.10 mg/mL | 0.0763 |

### Example 5

The process for preparing PEG-IFN α-2b composition is the same as described in Example 1, wherein the cryoprotectant used is PVP 4000 and buffer used is L-Histidine and L-Arginine. The quantities of the excipients along with the PEG-IFN α-2b is provided in table 5.

TABLE 5
Unit formula for the composition of PEG-IFN α-2b in the stability studies

| | | | Molar Conc. |
|---|---|---|---|
| Ingredients | Qty./vial | Conc. | (mM) |
| PEG-IFN α-2b | 0.12 mg | 0.16 mg/mL | 0.005 |
| L-Histidine | 0.85 mg | 1.15 mg/mL | 7.43 |
| L-Arginine hydrochloride | 10.54 mg | 14.24 mg/mL | 67.58 |
| PVP 4000 | 59.20 mg | 80.00 mg/mL | 2.0 |
| Polysorbate 80 | 0.074 mg | 0.10 mg/mL | 0.0763 |

### Example 6

The formulated PEG-IFN α-2b bulk was filled aseptically in vials (0.74 ml/vial) and was half stoppered with two-legged bromobutyl stoppers before loading into the lyophilizer. The lyophilization cycle used for the formation of cake is as mentioned below.

TABLE 6
Lyophiliztion cycle

| Stage | Temp (° C.) | Duration (min) | | Pressure |
|---|---|---|---|---|
| Freezing | 5.0 | 4 | hrs | Ambient |
| | −40.0 | 4 | hrs | Ambient |
| Annealing | −40.0 | 1 | hr | Ambient |
| | −30.0 | 1.5 | hrs | Ambient |
| | −40.0 | 0.5 | hr | Ambient |
| 1° Drying | −40.0 | 4 | hrs | 100 mTorr |
| | −30.0 | 5 | hrs | 100 mTorr |
| | 5.0 | 7.5 | hrs | 100 mTorr |
| 2° Drying | 5.0 | 0.5 | hr | 50 mTorr |
| | 15.0 | 10 | hrs | 50 mTorr |
| | 30.0 | 10 | hrs | 50 mTorr |

After the completion of lyophilization cycle, the vials were stoppered inside lyophilizer and were then removed from lyophilizer. Vials were visually inspected for cake formation.

The vials containing the lyophilized composition were analyzed for stability.

The stability of the protein at various time points (0, 1, 4, 8, 12, 24 weeks) was determined at 5° C., 25° C. by checking the protein profile by Size exclusion and Ion exchange chromatography. Also pH, osmolality and moisture content were determined.

The stability studies have shown that the pharmaceutical composition is stable at 5° C. 25° C. for 6 months and 40° C. for 4 weeks. The stability studies of these compositions are on-going and the protein profile will be checked at respective time points using the same parameters mentioned earlier.

All patents, patent applications and publications cited in this application are hereby incorporated by reference in their entirety for all purposes to the same extent as if each individual patent, patent application or publication were so individually denoted.

Although certain embodiments and examples have been described in detail above, those having ordinary skill in the art will clearly understand that many modifications are possible in the embodiments and examples without departing from the teachings thereof.

1. A stabilized pharmaceutical composition comprising PEG-IFN α-2b and cryoprotectant selected from the group consisting of HPBCD, sucralose and PVP 4000.

2. The stabilized pharmaceutical composition of claim 1 further comprises a buffer.

3. The stabilized pharmaceutical composition of claim 2 wherein buffer is selected from the group comprising of phosphate-citrate buffer, phosphate buffer, citrate buffer, L-Histidine, L-Arginine hydrochloride, bicarbonate buffer, succinate buffer, citrate buffer, TRIS buffer, either alone or in combination.

4. A stabilized pharmaceutical composition comprising PEG-IFN α-2b, cryoprotectant selected from the group consisting of HPBCD, sucralose and PVP 4000, a buffer, a surfactant and optionally a tonicity agent; wherein the said composition is sterile and ready for parenteral administration having pH in the range of 4.0 to 8.0.

5. The stabilized pharmaceutical composition of claim 4, wherein the buffer is phosphate-citrate buffer.

6. The stabilized pharmaceutical composition of claim 4, wherein the buffer is L-Histidine, L-Arginine hydrochloride buffer.

7. The stabilized pharmaceutical composition of claim 4, wherein the surfactant is selected from the group comprising of polysorbates, dodecyl sulfate (SDS), Lecithin either alone or in combination.

8. The stabilized pharmaceutical composition of claim 4, wherein the tonicity agent is selected from a group of salts comprising of sodium chloride, potassium chloride, calcium chloride; group of saccharides comprising of mannitol, sucrose, glucose and their likes and/or amino acids comprising of arginine, cysteine, histidine and the like.

9. The stabilized pharmaceutical composition of claim 4 comprising PEG-IFN α-2b; cryoprotectant selected from the group consisting of HPBCD, sucralose and PVP 4000; buffer selected from phosphate-citrate buffer and L-Histidine, L-Arginine hydrochloride buffer and polysorbate 80 as a surfactant and optionally sodium chloride as a tonicity agent; wherein the said composition is sterile and ready for parenteral administration.

10. The pharmaceutical composition of claim 4, comprising 0.03 mg/ml to 2 mg/ml of PEG-IFN α-2b, about 10 mg/ml to 250 mg/ml HPBCD, about 1 mM to 100 mM of phosphate citrate buffer, about 0 mg/ml to 9 mg/ml sodium chloride and about 0.01 mg/ml to 1 mg/ml polysorbate 80 having pH in the range of 4.0 to 8.0.

11. The pharmaceutical composition of claim 4, comprising 0.03 mg/ml to 2 mg/ml of PEG-IFN α-2b, about 10 mg/ml to 150 mg/ml sucralose, about 1 mM to 100 mM of phosphate citrate buffer or about 1 mM to 100 mM of L-Histidine, L-Arginine hydrochloride, and about 0.01 mg/ml to 1 mg/ml polysorbate 80 having pH in the range of 4.0 to 8.0.

12. The pharmaceutical composition of claim 4, comprising 0.03 mg/ml to 2 mg/ml of PEG-IFN α-2b, about 10 mg/ml to 150 mg/ml PVP 4000, about 1 mM to 100 mM of L-Histidine, L-Arginine hydrochloride and about 0.01 mg/ml to 1 mg/ml polysorbate 80 having pH in the range of 4.0 to 8.0.

13. The composition of claim 1 wherein the composition is a powder, an aqueous composition, or a reconstituted liquid composition.

14. A kit comprising a composition of claim 1 and instructions for use of the said composition.

15. The Kit of claim 14, wherein the composition is liquid or lyophilized powder.

16. The kit of claim 14, wherein the composition is stored in a pre-filled sterile syringe or vial or cartridge.

17. A method for treating hepatitis C, hepatitis B or melanoma with microscopic or gross nodal involvement within 84 days of definitive surgical resection including complete lymphadenectomy comprising administering the pharmaceutical composition of claim 1.
